(19) United States
(12) Patent Application Publication
(10) Pub. No.: US 2005/0119546 A9
(43) Pub. Date: Jun. 2, 2005

(54) Systems and methods for locating blood vessels

(76) Inventor: Connell Reynolds, Fairburn, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/631,146

(22) Filed: Jul. 31, 2003

Related U.S. Application Data
(60) Provisional application No. 60/399,982, filed on Jul. 31, 2002.

(51) Int. Cl. A61B 5/05; A61B 8/00

(52) U.S. Cl. 600/407; 600/443

(57) ABSTRACT

A method for locating a blood vessel includes transmitting waves into a body part through which a blood vessel runs, detecting reflections of the waves, determining a location of the blood vessel responsive to detecting the reflections of the waves, and providing a visual indication at a location that is adjacent to the blood vessel.

### BACKGROUND

A medical professional is often charged with the task of injecting a medicine or drug into a patient. This task may be complicated if an appropriate blood vessel for receiving an injection is not detected by the medical professional. A blood vessel may be undetectable for various reasons, including for example, if the patient has very low blood pressure, is obese, or is very young. Detecting a blood vessel through which to provide a patient with a needed drug or medicine may save a patient's live. Conversely, failing to detect such a blood vessel can prevent the patient from receiving a life- saving medicine or drug. Prior art systems and methods have not enabled emergency medical professionals to quickly and accurately determine a precise location where a drug or medicine may be injected into a patient in cases where blood vessels are not visible to the naked eye. Therefore, there exists a need for improved systems and methods for locating blood vessels.

### SUMMARY

Systems and methods for locating a blood vessel are disclosed. An embodiment of a method for locating a blood vessel includes transmitting waves into a body part through which a blood vessel runs, detecting reflections of the waves, determining a location of the blood vessel responsive to detecting the reflections of the waves, and providing a visual indication at a location that is adjacent to the blood vessel.

An embodiment of a system for locating a blood vessel includes a transmitter configured to transmit waves into a body part through which a blood vessel runs, a receiver configured to receive reflections of the waves transmitted by the transmitter, a processor that is programmed to determine a location of the blood vessel responsive to the receiver receiving the reflections of the waves, and a display device that is configured to provide a visual indication at a location that is adjacent to the blood vessel responsive to the processor determining the location of the blood vessel.

Other systems, methods, features and/or advantages will be or may become apparent to one with skill in the art upon examination of the following figures and detailed description. It is intended that all such additional systems, methods, features, and/or advantages be included within this description and be protected by the accompanying claims

### BRIEF DESCRIPTION OF THE DRAWINGS

FIG. 1 is a simplified block diagram of an embodiment of the blood-vessel locating-system.

FIG. 2A is a flow chart illustrating a blood-vessel locating-method according to an embodiment of the present invention.

FIG. 2B is a flow chart illustrating an exemplary method of the blood-vessel locating-method.

FIG. 3A is a schematic diagram depicting a frontal view of a strap-mounted blood-vessel locating-system.

FIG. 3B is a schematic diagram depicting a plan view of the strap-mounted blood-vessel locating-system.

FIG. 4A is a schematic diagram depicting a frontal view of a strap-mounted blood-vessel locating-system.

FIG. 4B is a schematic diagram depicting a plan view of the strap-mounted blood-vessel locating-system.

FIG. 5 is a schematic diagram illustrating the strap-mounted blood-vessel locating-system being used to indicate the location of a blood vessel that is flowing through a patient's arm.

### DETAILED DESCRIPTION OF THE PREFERRED EMBODIMENTS

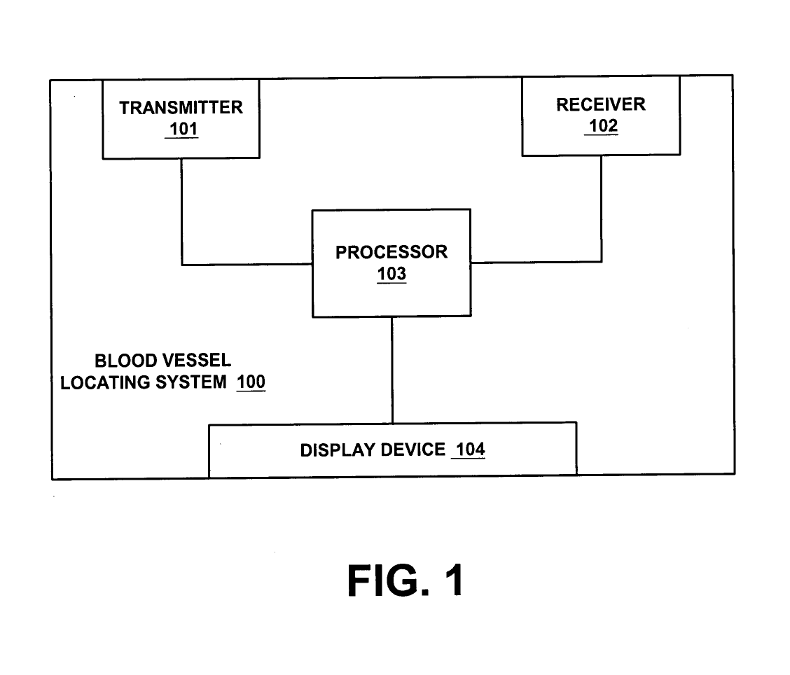

FIG. 1 is a simplified block diagram of an embodiment of the blood-vessel locating-system 100. The locating system 100 includes a transmitter 101, a receiver 102, and a display device 104 that are coupled to a processor 103. In a preferred embodiment, two or more receivers 102 are included in the blood-vessel locating-system 100.

The display device 104 may comprise, for example, LEDs, a laser pointer and/or an LCD display. One advantage of using LCDs is that they can be easily read in bright light and in the dark (with the addition of a back light). Custom LCD displays enable the use of graphic icons, text, gauges, and indicators.

In one embodiment, the transmitter 101 transmits ultrasound waves which reflect off the interior of a body part (e.g., a patient's arm) and which are received by the receiver 102. The receiver 102 converts the received ultrasound waves into electric signals and sends the electric signals to the processor 103.

The processor 103 analyzes the electric signals received from the receiver 102 to determine the location of one or more blood vessels. The processor 103 then sends signals to a display device 104 causing the display device 104 to provide one or more visual indications at one or more locations that are adjacent to the respective detected blood vessel(s). The processor 103 may be configured to process buffered signals, and may have a DSP core or may interface with a DSP processor. Two or more processors 103 may alternatively be used to enable operation of the blood-vessel locating-system 100.

In one embodiment, the blood-vessel locating-system 100 samples received signals at or above the corresponding Nyquest rate and applies a fast Fourier transform (FFT) to get the signals into the frequency domain. The blood-vessel locating-system 100 then analyzes the data with appropriate algorithms to determine blood vessel locations.

According to another embodiment of the invention, undersampling (also called bandpass sampling), allows the sampling frequency to be up to three hundred times less than that used in FFT. Undersampling works because loss of aliased frequency components of the input signal is avoided by properly selecting a sampling frequency and bandwidth for the input signals.

According to yet another embodiment of the invention, demodulation is used to reduce the required sampling frequency and buffer size. Most common demodulator designs use quadrature demodulation to get a complex signal that requires two analog mixers per channel. However, the blood-vessel locating-system 100 may be implemented using only one mixer per channel since the direction of blood flow is typically irrelevant.

The blood-vessel locating-system 100 may use a linear array of receivers 102 to locate a vessel. An array of receivers 102 may be placed over a vessel by an examiner. If the array of receivers 102 is centered over the vessel, the signals received by the receivers 102 on either side of the transmitter will match. If the array of receivers 102 is not centered, then the received signals will not match. Signals may be processed to show a spike representing the received Doppler shift with respect to time. Trigonometric algorithms may be used to derive the location and depth of a vessel.

Most medical ultrasound units operate at approximately 3-10 MHz in transcutaneous applications. Frequencies as high as 50 MHz have been used with ultrasound catheters. Lower frequencies penetrate tissue further but offer lower resolution. In one implementation, the blood-vessel locating-system 100 may use, for example, a frequency of about 8 MHz. Choosing one frequency or a narrow band of frequencies may enable a reduction in the size, complexity and cost of the blood-vessel locating-system 100.

An algorithm or method used to determine vessel location may be selected based on the layout of an array of receivers 102. Given a linear array of receivers 102, each receiver may provide respective data representing the magnitude of the received Doppler shift with respect to time. Regardless of whether the data is the product of a FFT or an analog signal produced by a demodulator, the data may be processed to determine the presence of Doppler shift with respect to time for each receiver (e.g., using trigonometric measures).

The blood-vessel locating-system 100 preferably uses continuous wave (CW) and/or pulse wave (PW) Doppler ultrasound with a demodulation circuit having suitable analog to digital converter (ADC). The Receiver 102 is preferably dampened to reduce signal noise and design complexity.

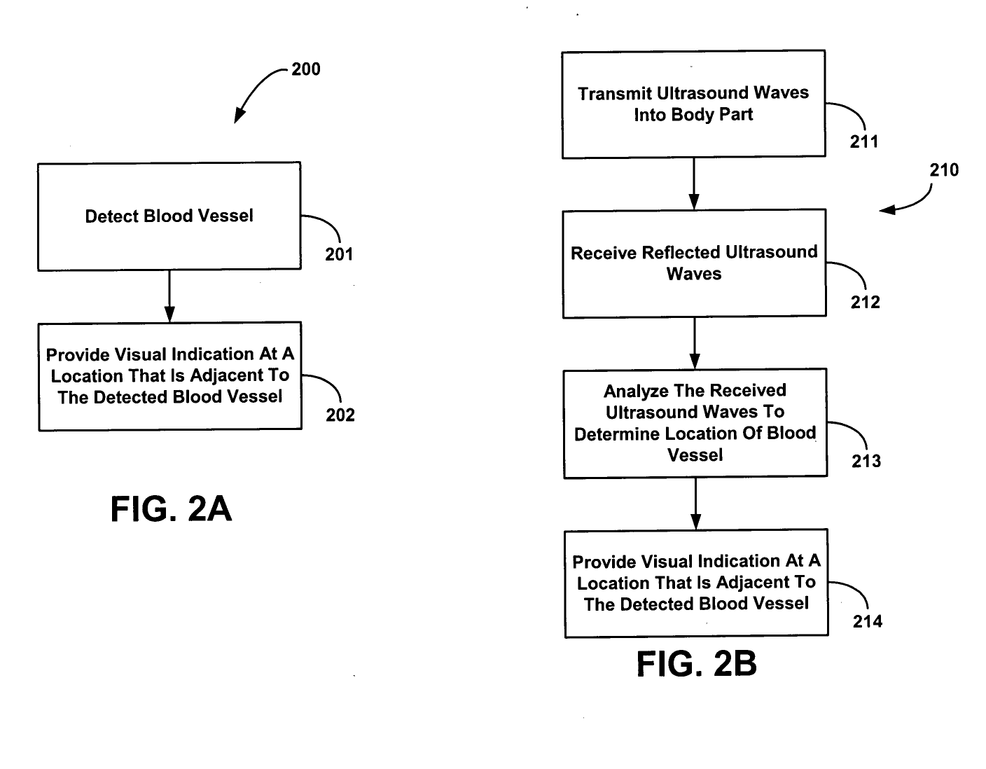

FIG. 2A is a flow chart illustrating a blood-vessel locating-method 200 according to an embodiment of the present invention. In step 201, a blood vessel is detected (e.g., using ultrasound, magnetic, or optical waves). Then, in step 202, a visual indication is provided at a location that is in the vicinity of and preferably adjacent to the detected blood vessel. As a result, a medical technician is able to quickly determine where to inject a patient with a drug or medicine. If several blood vessels are detected, then a plurality of visual indications may be provided at locations that are adjacent to the respective blood vessels. Alternatively, a visual indication is provided at a location that is adjacent to the blood vessel that is determined to have the highest rate of blood flow.

FIG. 2B is a flow chart illustrating an exemplary method 210 of the blood-vessel locating-method 200. In step 211, ultrasound waves are transmitted into a body part (e.g., a patient's arm). In step 212, ultrasound waves that reflect off the interior of the body are received. After the ultrasound waves are received, they are analyzed to determine the location of a blood vessel in the body part, as indicated in step 213. In step 214, a visual indication is provided at a location that is in the vicinity of and preferably adjacent to the detected blood vessel. In an alternative embodiment, light waves or other energy waves may be transmitted, received, and analyzed to help determine the location of a blood vessel.

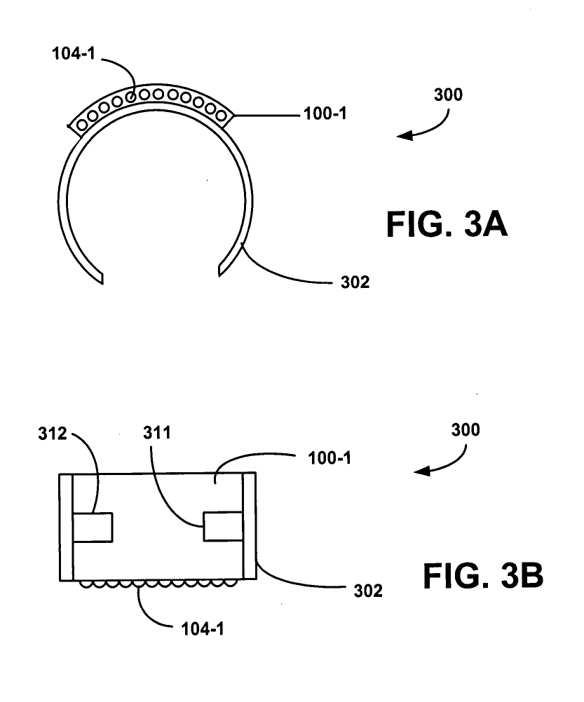

FIGS. 3A and 3B are schematic diagrams depicting a frontal view and a plan view, respectively, of a strap-mounted blood-vessel locating-system 300. The strap-mounted blood-vessel locating-system 300 includes a blood-vessel locating-system 100-1 and a strap 302 for mounting the locating system 100-1 on a patient (e.g., on a patient's arm). The strap 302 may comprise, for example, a belt, adhesive, and/or a hook-and-loop mechanism. Any suitable fastening means other than the strap 302 may alternatively be used. The blood-vessel locating-system 100-1 includes a plurality of light indicators 104-1 (e.g., light emitting diodes (LED's)). A light indicator 104-1 that is located closest to a detected blood vessel may emit light to indicate the location of the blood vessel.

As shown in FIG. 3B, the blood-vessel locating-system includes display devices 311 and 312, each of which may be, for example, a liquid crystal display (LCD). The display device 311 may be used to display a numeral and/or a letter indicating the depth of a detected blood vessel, which may be, for example, between 1 and 30 mm. The display device 312 may be used to display a numeral and/or a letter indicating the blood flow rate in a detected blood vessel.

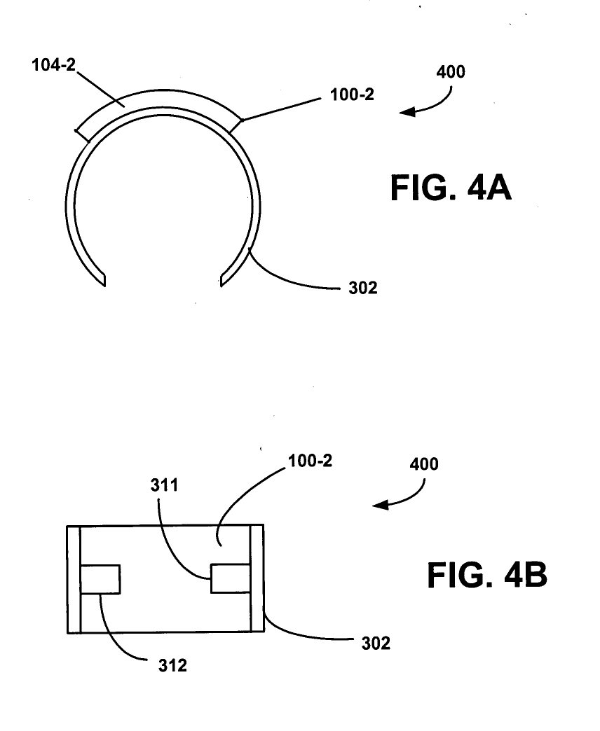

FIGS. 4A and 4B are schematic diagrams depicting a frontal view and a plan view, respectively, of a strap-mounted blood-vessel locating-system 400. The strap-mounted blood-vessel locating-system 400 includes a blood-vessel locating-system 100-2 and a strap 302 for mounting the blood-vessel locating-system 100-2 on a patient. The system 400 includes a display device 104-2. The display device 104-2 may be, for example, a liquid crystal display (LCD). A portion of the display device 104-2 that is located closest to a detected blood vessel may darken or lighten (depending on a desired implementation) to indicate the location the blood vessel. As shown in FIG. 4B, the strap-mounted blood-vessel locating-system 400 includes display devices 311 and 312, each of which may function as discussed above in reference to FIG. 3B.

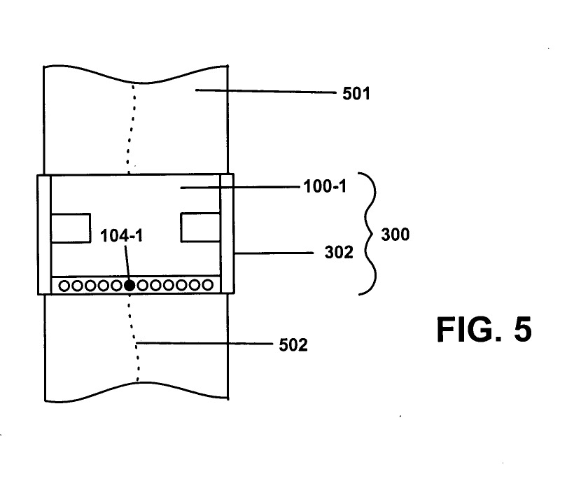

FIG. 5 is a schematic diagram illustrating the strap-mounted blood-vessel locating-system 300 being used to indicate the location of a blood vessel 502 that is flowing through a patient's arm 501. As shown in FIG. 5, the strap 302 is used to mount the locating system 100-1 onto the patient's arm 501. When the locating system detects a blood vessel, the light emitter 104-1 emits light to indicate that the detected blood vessel 502 is located immediately below the light emitter 104-1.

It should be emphasized that the above-described embodiments of the present invention are merely possible examples, among others, of the implementations, setting forth a clear understanding of the principles of the invention. Many variations and modifications may be made to the above-described embodiments of the invention without departing substantially from the principles of the invention. All such modifications and variations are intended to be included herein within the scope of the disclosure and present invention and protected by the following claims.

1. A method for locating a blood vessel, comprising the steps of:
transmitting waves into a body part through which a blood vessel runs;
detecting reflections of the waves;
determining a location of the blood vessel responsive to detecting the reflections of the waves; and
providing a visual indication at a location that is adjacent to the blood vessel, the visual indication being provided via a device that is attached to the body part.

2. The method of claim 1, wherein the waves comprise at least one of sound waves, optical waves, and magnetic waves.

3. The method of claim 1, wherein the step of providing the visual indication comprises turning on a light.

4. The method of claim 1, wherein the step of providing the visual indication comprises changing a brightness of a portion of a display device.

5. A system comprising:
a transmitter configured to transmit waves into a body part through which a blood vessel runs;
a receiver configured to receive reflections of the waves transmitted by the transmitter;
a processor that is programmed to determine a location of the blood vessel responsive to the receiver receiving the reflections of the waves; and
a display device that is configured to provide a visual indication at a location that is adjacent to the blood vessel responsive to the processor determining the location of the blood vessel;
wherein the system is configured to be attached to the body part.

6. The system of claim 5, wherein the waves comprise at least one of sound waves, optical waves, and magnetic waves.

7. The system of claim 5, wherein the display device comprises a liquid crystal display (LCD).

8. The system of claim 5, wherein the display device comprises light-emitting diodes (LEDs).

9. A system for locating a blood vessel, comprising the steps of:
means for transmitting waves into a body part through which a blood vessel runs;
means for detecting reflections of the waves;
means for determining a location of the blood vessel responsive to detecting the reflections of the waves;
means for providing a visual indication at a location that is adjacent to the blood vessel; and
means for attaching the system to the body part.

10. The system of claim 9, wherein the waves comprise at least one of sound waves, optical waves, and magnetic waves.

11. The system of claim 9, wherein the means for providing a visual indication comprises a liquid crystal display (LCD).

12. The system of claim 9, wherein the means for providing a visual indication comprises light-emitting diodes (LEDs).
